(19) United States
(12) Patent Application Publication
(10) Pub. No.: US 2020/0397471 A1
(43) Pub. Date: Dec. 24, 2020

(54) VASCULAR SHEATH WITH MULTIPLE OUTLETS AND METHODS OF USING SAME

(71) Applicant: Farajallah Hanna al-kass, Glendale, CA (US)

(72) Inventor: Farajallah Hanna al-kass, Glendale, CA (US)

(21) Appl. No.: 16/450,097

(22) Filed: Jun. 24, 2019

(51) Int. Cl. A61B 17/34; A61B 5/00

(52) U.S. Cl. CPC A61B 17/3423; A61B 2017/00331; A61B 5/6852; A61B 17/3462

(57) ABSTRACT

An introducer includes a hub having a port, and a flexible cylindrical sheath coupled to the hub and having a wall defining a lumen longitudinally extending therethrough, the lumen terminating in a first outlet opposite the port, the sheath further including a second outlet defined in the wall and in communication with the lumen.

### FIELD OF THE DISCLOSURE

The present disclosure relates to catheters and introducers for use in medical settings. More particularly the present disclosure relates to introducer and catheter devices and methods to help reduce operation time.

### BACKGROUND OF THE DISCLOSURE

Certain medical procedures may require the use of devices for intravascular access. In some cases, catheters are used to treat a patient or to deliver radiopaque media to select sites in the vascular system in conjunction with the routine diagnostic procedures. In some cases, a patient may need imaging in multiple vessels, which may require multiple procedures.

In addition to causing discomfort to the patient, a large number of procedures also increases the risk of infection or other complications. Additionally, performing multiple procedures unnecessarily increases the cost of patient care and wastes time of a trained physician.

### SUMMARY OF THE DISCLOSURE

In some examples, an introducer includes a hub having a port, and a flexible cylindrical sheath coupled to the hub and having a wall defining a lumen longitudinally extending therethrough, the lumen terminating in a first outlet opposite the port, the sheath further including a second outlet defined in the wall and in communication with the lumen.

### BRIEF DESCRIPTION OF THE DISCLOSURE

Various embodiments of the presently disclosed devices are shown herein with reference to the drawings, wherein:

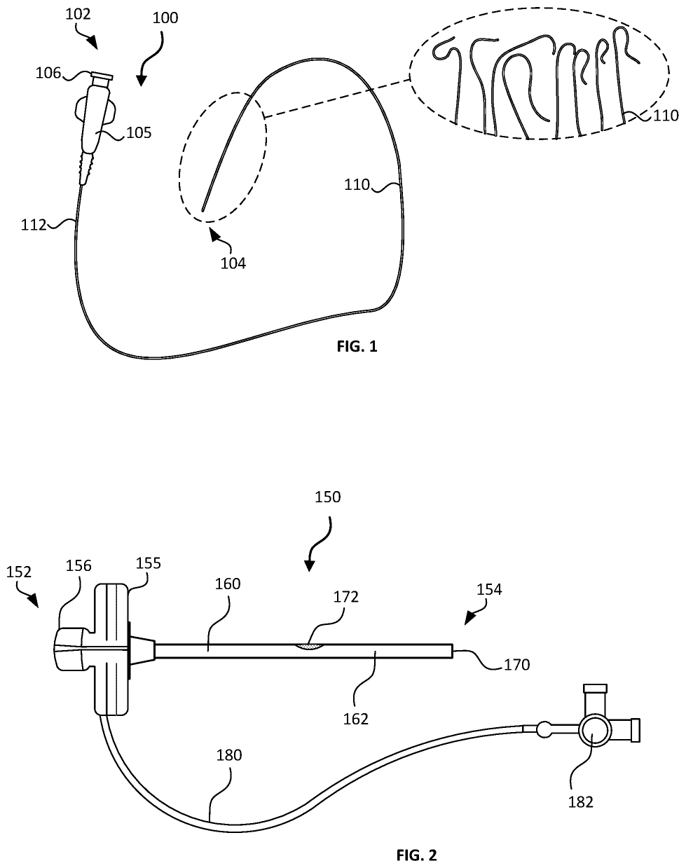

FIG. 1 is a schematic top view of a diagnostic catheter and detailed view of certain shaft configurations;

FIG. 2 is a schematic side view of an introducer according to one embodiment of the present disclosure;

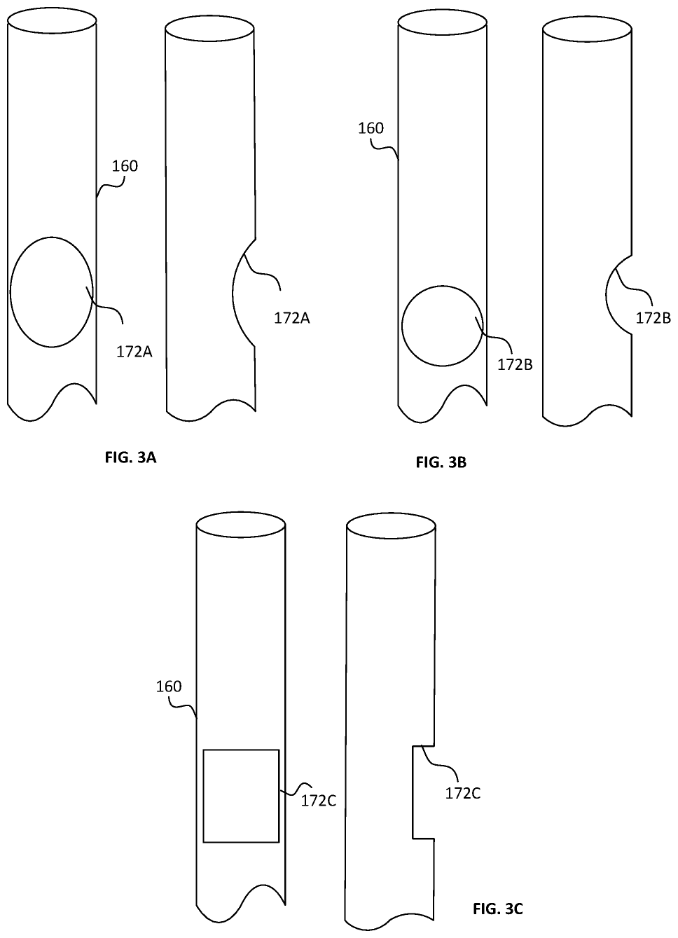

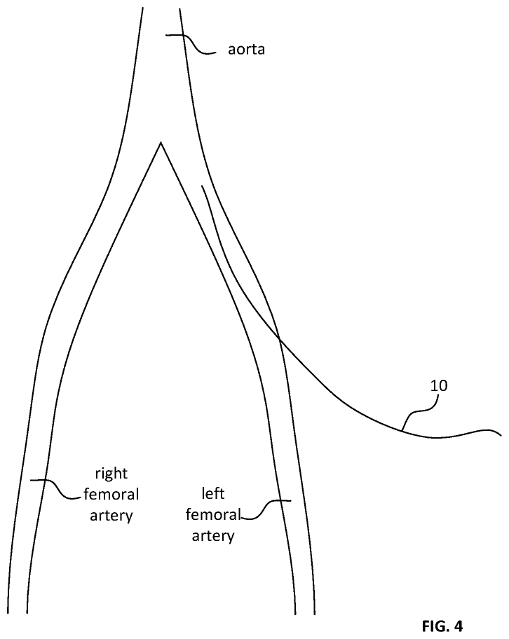

FIGS. 3A-C are schematic detailed views of secondary outlets formed in a sheath;

FIGS. 4-8 are schematic representations showing the use of the introducer of FIG. 2 in one position through a first outlet and a second position through a second outlet;

FIG. 9 is a schematic representation showing the use of the introducer adjacent the profunda femoris; and

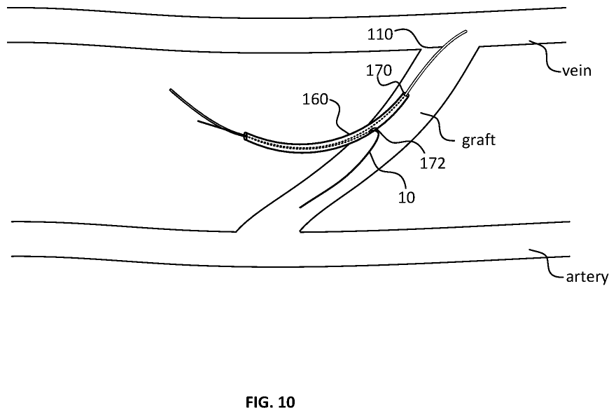

FIG. 10 is a schematic representation showing the use of the introducer adjacent a dialysis graft.

Various embodiments of the present invention will now be described with reference to the appended drawings. It is to be appreciated that these drawings depict only some embodiments of the invention and are therefore not to be considered limiting of its scope.

### DETAILED DESCRIPTION

Despite the various improvements that have been made to catheter and introducer devices and their methods of use, conventional devices suffer from some shortcomings as described above. There therefore is a need for further improvements to the devices, systems, and methods of making and using catheters and introducers within the body. The present introducers may be used with diagnostic catheters as well as intervention such as balloon catheters, suction thrombectomy catheters, and atherectomy catheters. Among other advantages, the present disclosure may address one or more of these needs.

FIG. 1 shows one example of a diagnostic catheter 100. Catheter 100 extends between proximal end 102 and distal end 104, and includes a hub 105 having a port 106. Hub 105 may be formed of a polymeric material, such as polyurethane or other suitable material. Catheter 100 further includes a flexible shaft 110 having a lumen 112, the shaft extending from the hub toward the distal end of the catheter. Lumen 112 and port 106 are in communication with one another such that a guidewire (not shown) may be inserted through the entire device from one end to the other. Shaft 110 may be pre-formed into a variety of shapes as shown in the detailed view and may have a shape memory so that it returns to the pre-formed shape absent an external force. The shape of the shaft 110 may be chosen based on the intended use and the patient anatomy. The shaft 110 may be visible under fluoroscopy and may include materials such as braided metals to increase flexibility and steerability while decreasing kinking. Catheter 100 may be advanced over a guidewire (not shown) through port 106 and lumen 112 of shaft 110. Additionally, catheter 100 is designed to deliver radiopaque media to a site within the vasculature.

Catheter 100 may be used in conjunction with, and inserted through, an introducer 150 (FIG. 2). Introducer 150 may generally extend between a first end 152 and a second end 154 and include a body 155 and sheath 160. Body 155 may be formed of a polymeric material similar to that of hub 105, and may have an opening extending therethrough from the first end 152 to the sheath and in communication with a lumen 162 of the sheath. Sheath 160 may be in the shape of a substantially cylindrical tube having a wall and may be flexible. Sheath 160 may terminate on one end in a first circular outlet 170 in communication with lumen 162. Sheath 160 may also include additional outlets, such as a second outlet 172 disposed approximately halfway along the longitudinal length of the sheath and formed on a wall of the sheath 160. The position of the second outlet may depend on the procedure and intended used, as well as the length of introducer 150. In some examples, a relatively short introducer is used (e.g., 10-13 cm). In other examples, a relatively long introducer is used (e.g., 75-100 cm). In some examples, the position of the second outlet may be selected at any level along the shaft. In at least some examples, the second outlet 172 may be approximately, 2 cm to 10 cm from the first outlet, or more specifically it may be from 3 cm to 4 cm from the first outlet 170.

Second outlet 172 may be oriented perpendicular to first outlet 170 and may be 2 French to 8 French in size. The size of second outlet may vary, and may be related to the size of the introducer. In at least some examples, the secondary outlet is one French smaller than the size of the introducer (e.g., a 5 French introducer may have a 4 French secondary outlet). Alternatively, the secondary outlet may be 2 French smaller than the size of the introducer (e.g., a 5 French introducer may have a 3 French secondary outlet).

In at least some examples, second outlet 172 may be an oval-shaped outlet 172A (FIG. 3A). In at least some examples, second outlet may be a circular outlet 172B (FIG. 3B). Alternatively, second outlet may be formed as slit 172C (FIG. 3C) in the wall of the sheath, or may have any other suitable shape. Optionally, second outlet 172 may be covered by a resealable septum, such as silicone, so that the outlet 172 is sealed and water-tight when a catheter is not advanced therethrough.

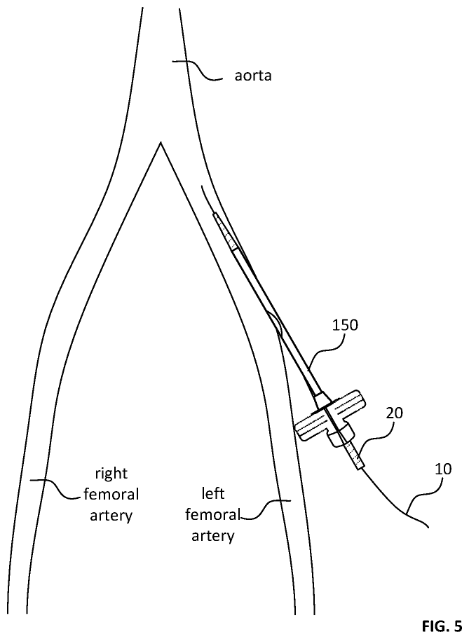

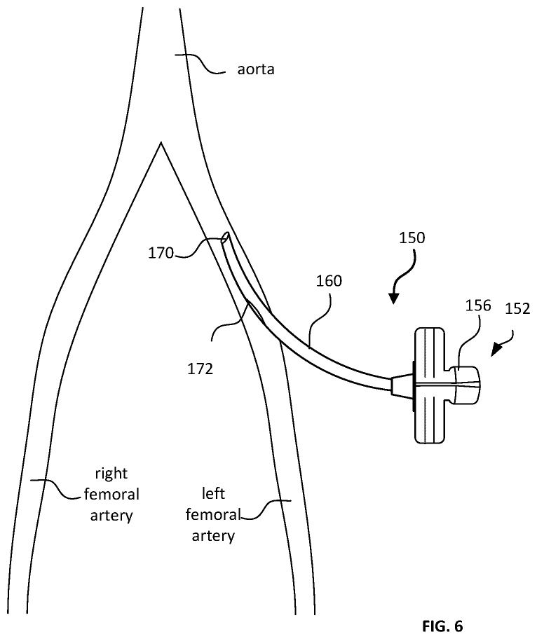

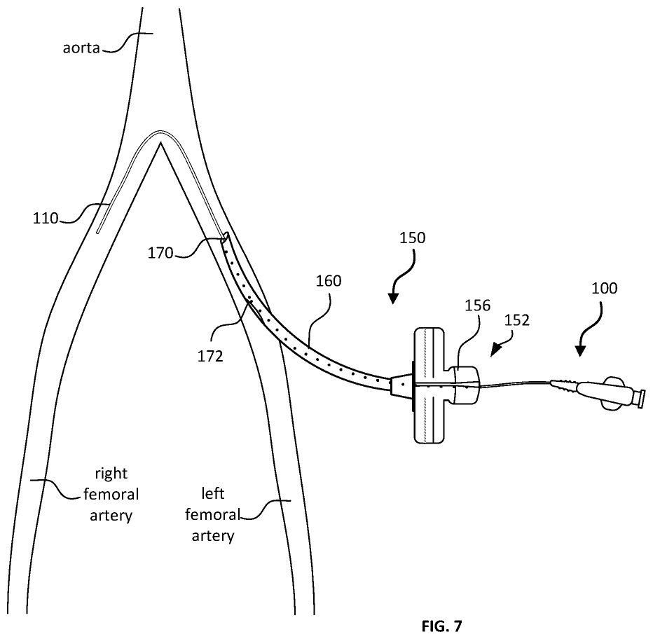

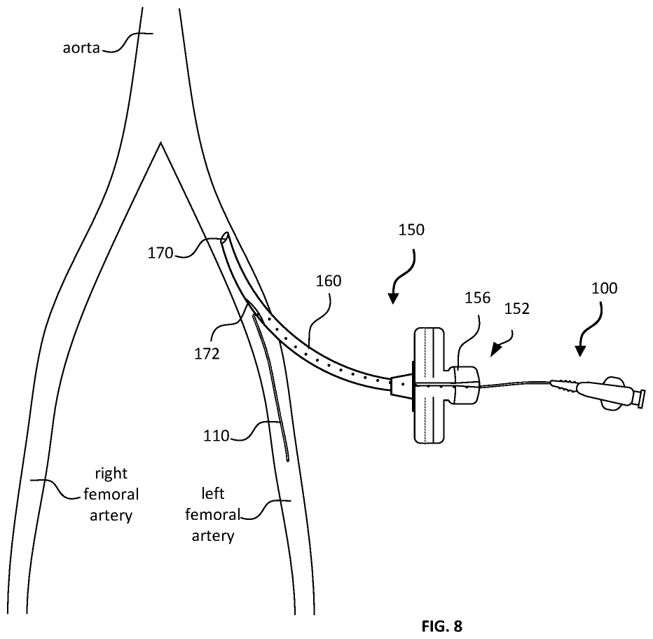

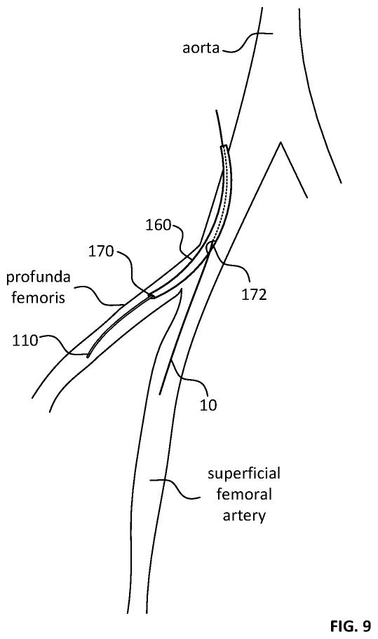

FIGS. 4-8 are schematic representations showing the use of the introducer of FIG. 2 in one position through a first outlet and subsequently in a second position through a second outlet. In use, an incision may be made in the patient's leg and a guidewire 10 may be inserted through the incision and passed into the left or right femoral artery (FIG. 4). A stiffened conical stylet may be inserted through the introducer 150 through the sheath of the introducer and the introducer may be advanced into position over the guidewire 10 (FIG. 5). With the introducer 150 in position at least partially within the artery, the stylet 20 and guidewire 10 may be removed, leaving the introducer in place with the sheath of the introducer being positioned within the femoral artery and oriented toward the aorta, the second outlet of the introducer being disposed within the femoral artery (FIG. 6). With the stylet removed, the introducer may be better adjusted due to its flexibility. A catheter, such as diagnostic catheter 100, may be advanced through introducer 150 and the shaft 110 of catheter 100 may pass through the body and lumen of sheath 160 of the introducer, exit first outlet 170 and be steered to the opposing femoral artery (i.e., the femoral artery opposite the site of incision) and imaging or treatment may be carried out (FIG. 7). When the imaging or treatment is complete, the catheter may be removed, leaving the introducer in place, and a second (or same catheter) may be advanced through the introducer 150 and exit through secondary outlet 172, the shaft 110 of the catheter being steered back down within the same femoral artery closer to the patient's lower extremities. A second imaging or treatment may be performed within the second femoral artery (FIG. 8). In this manner, a single incision and insertion of introducer 150 is performed, and both femoral arteries or other arteries within the body may be imaged and/or treated as desired without having to access multiple arteries separately and created multiple incisions in the patient's body.

It will be understood that a catheter may be chosen based on the desired path and the predetermined outlet of the introducer. For example, a catheter having a preformed shape with a 90-degree or perpendicular turn at the distal end may be chosen when advancement through the second outlet of the introducer is desired. Alternatively, a catheter with a different shape (e.g., more linear or with a smoother curve) may be chosen when advancement through the first outlet is intended. For the purpose of doing diagnostic and/or interventional procedures through secondary outlet, a 90-degree catheter is desirable and may be suitable in being introducer through the acute angle between the direction of the introducer and the opposing vessel that being or will be treated. Additionally, it will be understood that that though use of the introducer has been shown in connection with imaging/treatment of the femoral arteries, the same may be performed at other sites within the patient's body. Moreover, the sequence of treatments may be chosen by the patient as desired starting with the closest artery first, or the farthest artery first.

FIG. 9 illustrates another method of using the introducer for antegrade vascular access at the femoral artery. As shown, sheath 160 of the introducer is inserted into the femoral artery. During this initial delivery, the introducer may end up either in the profunda femoris artery or superficial femoral artery. If, for example, the introducer ended up in the profunda femoris, then the physician will spend time to try to negotiate the introducer to go into the superficial femoral artery to diagnose and/or treat it. Negotiating this turn may be challenging, especially if the antegrade entry into the common femoral artery is near the bifurcation of profunda femoris and superficial femoral arteries. In some instances, in the process of pulling back the introducer to go from the profunda femoris to the superficial femoral artery, the sheath might slip out of the common femoral artery and lose the arterial access. Losing access may impose increased risks to the patient and increase radiation to patient and the physician. Instead, by following this method and using the dual-outlet introducer, the risk of losing access is reduced, because while the guidewire is advanced into the superficial femoral artery through the secondary outlet, most of the introducer is still in the profunda femoris. Once the guidewire is in the superficial femoral artery, the introducer will be slightly retracted and re-advanced over the guidewire in the superficial artery. This will results in a decreased chance of loss of access, decreased dose of radiation to the patient and staff, and decreased chance of complications due to multiple arterial access.

FIG. 10 illustrates another method of using the introducer, this time in the context of treating a vein or an artery connected together by a dialysis graft of fistula. In the example shown, an introducer shaft 160 may inserted into a graft, and a guidewire may exist the secondary outlet 172 toward an artery, while a catheter shaft 110 exits the first outlet 170 toward the vein. It will be understood that the opposite configuration is possible with the guidewire being advanced toward the vein and exiting from the first or second outlet, while the catheter is advanced toward the artery and exists from the outlet not utilized by the guidewire.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

It will be appreciated that the various dependent claims and the features set forth therein can be combined in different ways than presented in the initial claims. It will also be appreciated that the features described in connection with individual embodiments may be shared with others of the described embodiments.

What is claimed is:

1. A introducer comprising:
a hub having a port; and
a flexible cylindrical sheath coupled to the hub and having a wall defining a lumen longitudinally extending therethrough, the lumen terminating in a first outlet opposite the port, the sheath further including a second outlet defined in the wall and in communication with the lumen.

2. The introducer of claim 1, wherein the second outlet is disposed approximately halfway along the length of the sheath.

3. The introducer of claim 1, wherein the second outlet is disposed closer to the first outlet than the hub.

4. The introducer of claim 1, wherein the second outlet is disposed closer to the hub than the first outlet.

5. The introducer of claim 1, wherein the second outlet has a same shape and a same size as the first outlet.

6. The introducer of claim 1, wherein the second outlet is circular or oval.

7. The introducer of claim 1, wherein the second outlet is covered by a self-sealing septum.

8. The introducer of claim 1, wherein the first outlet and the second outlet are perpendicular to one another.

9. A kit comprising:
the introducer of claim 1; and
a catheter having a shaft sized and arranged to fit within the lumen of the sheath.

10. The kit of claim 9, wherein the shaft includes a shape memory material that is preformed and includes a 90-degree bend adjacent a distal end.

11. A method of treating a patient comprising:
providing an introducer having a hub including a port, and a flexible cylindrical sheath coupled to the hub and having a wall defining a lumen longitudinally extending therethrough, the lumen terminating in a first outlet opposite the port, the sheath further including a second outlet defined in the wall and in communication with the lumen;
providing a catheter having a shaft sized and arranged to fit within the lumen of the sheath; and
advancing the shaft of the catheter through one of the first outlet and the second outlet to a first site.

12. The method of claim 11, further comprising the step of making an incision in the patient's body and inserting a guidewire into the patient's femoral artery.

13. The method of claim 12, further comprising the step of inserting a stiffening stylet into the introducer and advancing the introducer and stylet over the guidewire until the introducer is in a first position within the patient's body where the second outlet is within the femoral artery.

14. The method of claim 13, further comprising the step of removing the stylet and guidewire while maintaining the introducer in the first position within the patient's body.

15. The method of claim 14, further comprising the step of withdrawing the shaft of the catheter after advancing it through one of the first outlet and the second outlet, and advancing the shaft of the catheter through a second of the first outlet and the second outlet to a second site.

16. The method of claim 11, wherein advancing the shaft includes advancing the shaft into one of the two femoral arteries.

17. The method of claim 11, further comprising the step of treating the patient at the first site.

18. The method of claim 11, wherein treating the patient includes a therapeutic procedure.

19. The method of claim 11, wherein treating the patient includes a diagnostic procedure.

20. The method of claim 15, further comprising the step of treating the patient at the second site.
